(19) United States
(12) Patent Application Publication
(10) Pub. No.: US 2016/0353992 A1
(43) Pub. Date: Dec. 8, 2016

(54) PULSE-PALPATING APPARATUS FOR PROXIMAL AND REMOTE PULSE PALPATION

(71) Applicant: Chien-Hsing HO, Taichung City (TW)

(72) Inventor: Chien-Hsing HO, Taichung City (TW)

(21) Appl. No.: 14/834,382

(22) Filed: Aug. 24, 2015

(30) Foreign Application Priority Data
Jun. 3, 2015 (TW) 104117895

(51) Int. Cl. A61B 5/00; A61B 5/024

(52) U.S. Cl. CPC A61B 5/0022; A61B 5/02444; A61B 5/0053; A61B 5/4854; A61B 5/6824; A61B 5/7271

(57) ABSTRACT

A pulse-palpating apparatus, comprising: a pulse-palpating device, including a pulse-palpating-end communicating means and a pressure-exerting detecting means which exerts a force on pulse taking positions on a human body via the detecting member, which detects a pulse information on the pulse taking positions; and a network-connected controlling device, including a network-connected controlling means and a controlling-end communicating means, in which the network-connected controlling means is configured to generate force-intensity-controlling information, and the controlling-end communicating means transmits the force-intensity-controlling information to the pulse-palpating-end communicating means via network transmission, and receives the pulse information of the detecting member via network transmission. The pulse-palpating apparatus is provided for users to perform self-pulse diagnosis, and for medical practitioners to perform remote pulse palpation on a subject via network.

### FIELD OF THE INVENTION

The present invention relates to a pulse-palpating apparatus, and more particularly to a pulse-palpating apparatus with remote pulse-palpating function.

### BACKGROUND OF THE INVENTION

To perform a pulse diagnosis in traditional Chinese medicine (TCM), a medical practitioner has a face-to-face meeting with a patient, and measures the patient's pulse so as to recognize the type, cause and changes of the disease according to information such as a arterial pulse rate and a pulse volume of the patient. Since pulse diagnosis require face-to-face consultations between the practitioners and the patients, elderly or disabled patients having difficulties seeking medical help on their own cannot obtain this kind of medical services. Therefore, it is necessary to provide a pulse-palpating apparatus with remote pulse-palpating function for medical practitioners to recognize the pulse signal of the patients.

### SUMMARY OF THE INVENTION

Accordingly, an object of the present invention is to provide a pulse-palpating apparatus which can be used for performing proximal and remote pulse palpation, by which the users can perform self-pulse palpation or by which medical practitioners can perform remote pulse palpation.

To solve the issues in the prior art, the present invention provides a pulse-palpating apparatus, comprising: a pulse-palpating device, including a pressure-exerting detecting means and a pulse-palpating-end communicating means which is in signal connection with the pressure-exerting detecting means, in which the pressure-exerting detecting means includes a pressure-exerting member and a detecting member in such a manner that after the pressure exerting member exerts a force on pulse taking positions on a human body, the detecting member detects pulse information on the pulse taking positions; and a network-connected controlling device, including a network-connected controlling means and a controlling-end communicating means, in which the network-connected controlling means is configured to generate force-intensity-controlling information, and the controlling-end communicating means transmits the force-intensity-controlling information to the pulse-palpating-end communicating means via network transmission between the pulse-palpating device and the network-connected controlling device, and receives the pulse information of the detecting member via network transmission, wherein the force exerted by the pressure-exerting member corresponds to the force-intensity-controlling information.

According to an embodiment of the present invention, the quantity of the pressure exerting-detecting means is three, and each pressure-exerting detecting means respectively corresponds to any one of the three pulse taking positions of Cun, Guan, and Chi on the human body.

According to an embodiment of the present invention, the pressure-exerting member includes a bulging body that contacts the detecting member, which contacts the three pulse taking positions of Cun, Guan, and Chi on the human body.

According to an embodiment of the present invention, the pressure-exerting detecting means is plural, and the pulse-palpating device includes a switching means that is electrically connected between the plurality of pressure-exerting detecting means and the pulse-palpating-end communicating means.

According to an embodiment of the present invention, the pulse-palpating device includes a signal converter that is in signal connection between the pressure-exerting detecting means and the pulse-palpating-end communicating means.

According to an embodiment of the present invention, the network-connected controlling means includes a press-sensing member that generates the force-intensity-controlling information according to a pressing manipulation.

According to an embodiment of the present invention, the controlling-end communicating means and the pulse-palpating-end communicating means are wirelessly connected.

According to an embodiment of the present invention, the network-connected controlling device includes an analyzing means that generates an analysis result according to the pulse information.

By the technical means adopted by the present invention, the pulse information and the force-intensity-controlling information is transmitted via network, which enables the pulse palpation to be performed with no distance limit. Accordingly, medical practitioners can perform remote pressure exerting and pulse information detecting via the network-connected controlling device, and therefore obtain the pulse information of the subject. In some embodiments of the present invention, the pressure-exerting detecting means can simulate the force of the pressing manipulation so as to detect the pulse information of the subject accurately.

Furthermore, the analyzing means analyzes the pulse information and generates the analysis result for the medical practitioner's reference. Also, with the analysis result, the subject becomes aware of his physical condition.

### BRIEF DESCRIPTION OF THE DRAWINGS

The technical means adopted by the present invention to achieve the above and other objects can be best understood by referring to the following detailed description of the preferred embodiments and the accompanying drawings.

FIG. 1 is a schematic diagram of a pulse-palpating apparatus according to a first embodiment of the present invention;

FIG. 2 is a schematic diagram of the pulse-palpating apparatus according to a second embodiment of the present invention;

FIG. 3 is a block diagram of the pulse-palpating apparatus according to the first embodiment of the present invention.

### DETAILED DESCRIPTION OF THE PREFERRED EMBODIMENTS

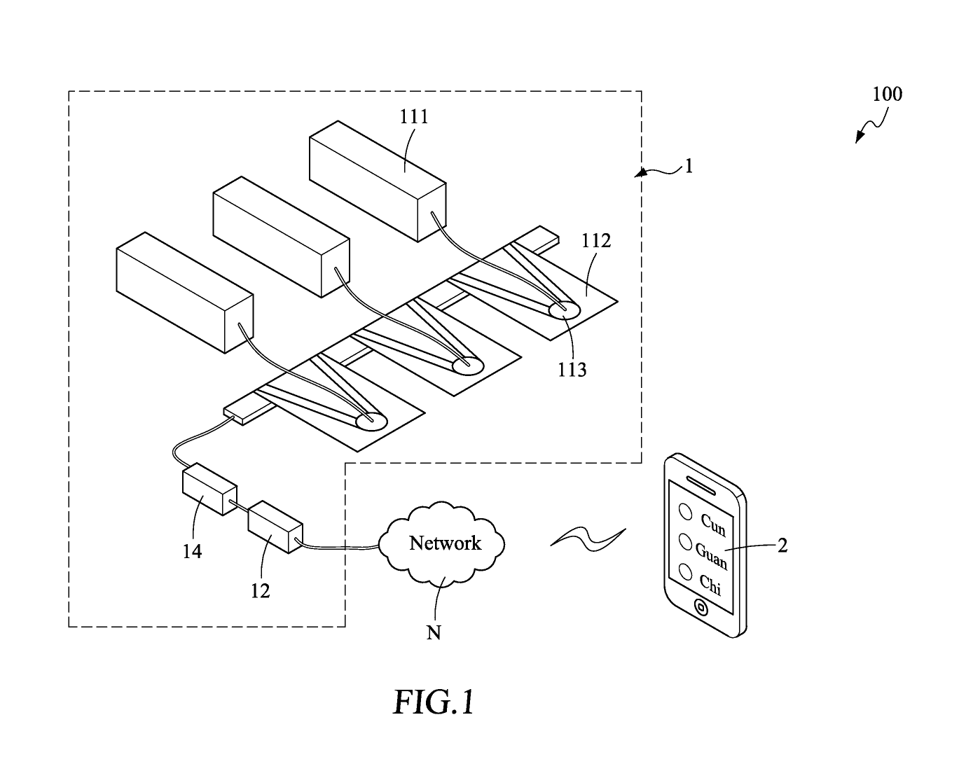

The preferred embodiments of the present invention are described in detail below with reference to FIG. 1 to FIG. 3. The description is used for explaining the embodiments of the present invention only, but not for limiting the scope of the claims.

As shown in FIG. 1, a pulse-palpating apparatus 100 according to an embodiment of the present invention comprises: a pulse-palpating device 1, including a pressure-exerting detecting means 11 and a pulse-palpating-end communicating means 12 which is in signal connection with the pressure-exerting detecting means 11, in which the pressure-exerting detecting means 11 includes a pressure-exerting member 111 and a detecting member 112 in such a manner that after the pressure exerting member 111 exerts a force on pulse taking positions on a human body, the detecting member 112 detects pulse information P on the pulse taking positions; and a network-connected controlling device 2, including a network-connected controlling means 21 and a controlling-end communicating means 22, in which the network-connected controlling means 21 is configured to generate force-intensity-controlling information C, and the controlling-end communicating means 22 transmits the force-intensity-controlling information C to the pulse-palpating-end communicating means 12 via network transmission between the pulse-palpating device 1 and the network-connected controlling device 2, and receives the pulse information P of the detecting member 112 via network transmission, wherein the force exerted by the pressure-exerting member 111 corresponds to the force-intensity-controlling information C.

In the pulse-palpating apparatus 100 according to the first embodiment of the present invention as shown in FIG. 1, the pressure exerting-detecting means 11 is plural. In the present embodiment, the quantity of the pressure exerting-detecting means 11 is three, and each pressure-exerting detecting means 11 respectively corresponds to any one of the three pulse taking positions of Cun, Guan, and Chi on the human body. The pressure-exerting member 111 of the pressure-exerting detecting means 11 is a micro pump, and the detecting member 112 which includes a preamplifier is a piezoelectric sensor.

In the pulse-palpating apparatus 100 according to the first embodiment of the present invention as shown in FIG. 1, the pressure-exerting member 111 includes a bulging body 113 that contacts the detecting member 112, which contacts the three pulse taking positions of Cun, Guan, and Chi on the human body. The size of the bulging body 113 is close to that of a fingertip. The bulging body changes its size according to the force-intensity-controlling information C. The change in size of the bulging body can change the force exerted against the pulse taking positions in intensity so as to simulate the pressing force of a medical practitioner performing the pulse palpation. Preferably, the pressure-exerting member 111 also includes a moving body (not shown in the Figs.) that connects with the detecting member 112 and the controlling-end communicating means 22. The moving body is used by the medical practitioner to perform an operation of moving the detecting member 112 at the network-connected controlling device 2 so as to adjust the position of the detecting member 112 to reach a precise pulse taking position.

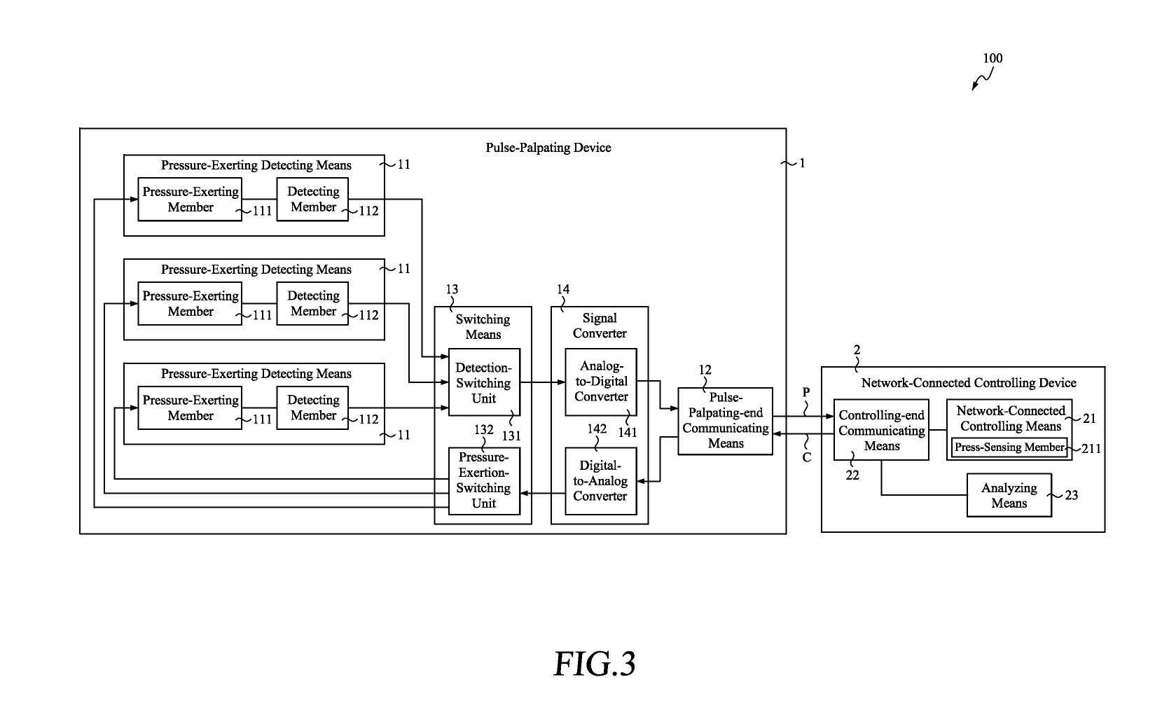

In the pulse-palpating apparatus 100 according to the first embodiment of the present invention as shown in FIG. 3, the pulse-palpating device 1 includes a switching means 13 that is electrically connected between the plurality of pressure-exerting detecting means 11 and the pulse-palpating-end communicating means 12. The switching means 13 includes a detection switching unit 131 and a pressure-exertion switching unit 132, wherein the detection switching unit 131 is a multiplexer that receives the pulse information P from the plurality of the detecting members and outputs a chosen pulse information P which is chosen from one of the plurality of the detecting members. The pressure-exertion switching unit 132 is a demultiplexer that chooses one of the output-ends which connects with the pressure-exerting member 111 and outputs the force-intensity-controlling information C.

In the pulse-palpating apparatus 100 according to the first embodiment of the present invention as shown in FIG. 3, the pulse-palpating device 1 includes a signal converter 14 that is in signal connection between the pressure-exerting detecting means 11 and the pulse-palpating-end communicating means 12. In the present embodiment, the plurality of the pressure-exerting detecting means 11 is in signal connection with one signal converter 14 via the switching means. In other embodiments, the plurality of the pressure-exerting detecting means 11 also can be in signal connection with the plurality of corresponding signal converters 14, and in signal connection with the pulse-palpating-end communicating means 12 via the switching means 13.

The signal converter 14 includes an analog-to-digital converter 141 and a digital-to-analog converter 142. The analog-to-digital converter 141 which includes a programmable gain amplifier (PGA) is in signal connection between the pressure-exerting detecting means 112 and the pulse-palpating-end communicating means 12, and converts analog pulse information P into digital signals so that the pulse information P in digital form can be transmitted by the pulse-palpating-end communicating means. The digital-to-analog converter 142 is in signal connection between the pulse-palpating-end communicating means and the pressure-exerting detecting means, and converts digital force-intensity-controlling information C into analog signals so as to control the force intensity of the pressure-exerting member 111.

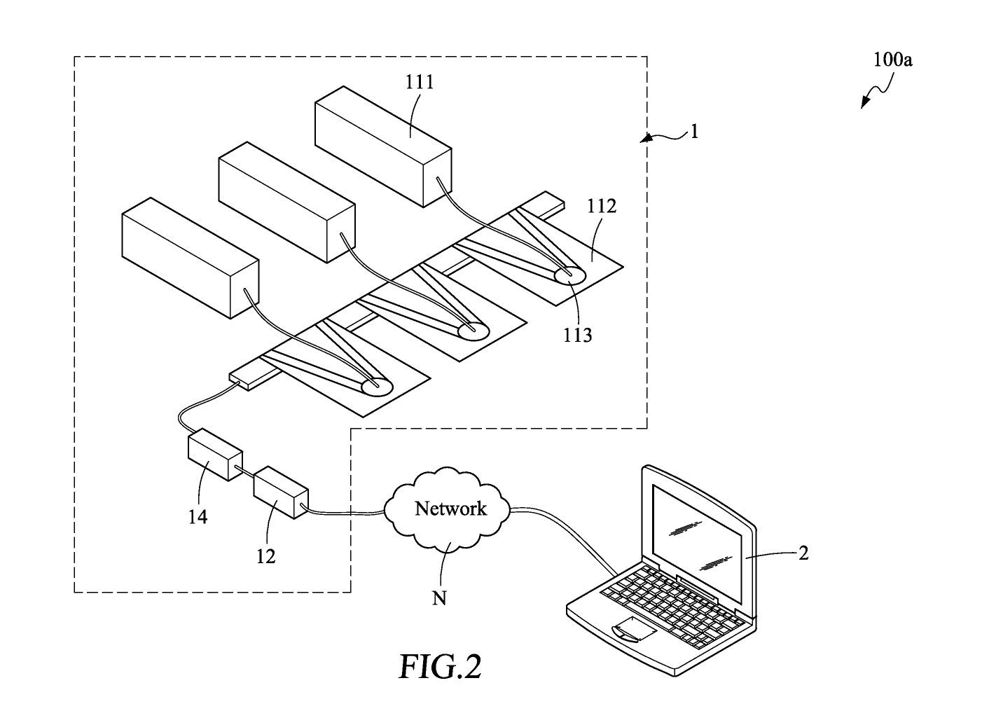

In the pulse-palpating apparatus 100 according to the first embodiment of the present invention as shown in FIG. 1, the network-connected controlling device 2 is a cell-phone, wherein the controlling-end communicating means 22 in the cell-phone and the pulse-palpating-end communicating means 12 at a proximal end or a remote end are in wireless network N connection. It goes without saying that the present invention is not limited to this. In the pulse-palpating apparatus 100a according to the second embodiment of the present invention as shown in FIG. 2, the network-connected controlling device 2 is a computer, wherein the controlling-end communicating means 22 in the computer and the pulse-palpating-end communicating means 12 at a proximal end or a remote end are in wired network N connection. The network-connected controlling device 2 also can be other smart device that performs mutual signal transmission with the pulse-palpating device 1 via wired network communication or wireless network communication, wherein the network-connected controlling device 2 uses an application (app) as a user interface for the medical practitioner to operate. The medical practitioner controls the pulse-palpating device 1 via the app and performs pulse-palpation on the subject.

In the pulse-palpating apparatus 100 according to the first embodiment of the present invention as shown in FIG. 1 and FIG. 3, the network-connected controlling means 21 includes a press-sensing member 211 that generates the force-intensity-controlling information C according to a pressing manipulation. In detail, the medical practitioner press the press-sensing member 211 at the Cun operating position, Guan operating position, and Chi operating position on the cell phone. The medical practitioner slides his/her fingertip upward on the press-sensing member 211 so as to generate a force-intensity-controlling information C, according to which the pressure-exerting member 111 exerts a “deep” force (a heavy press). Alternatively, the medical practitioner lightly presses his/her fingertips on the press-sensing member 211 so as to generate another force-intensity-controlling information C, according to which the pressure-exerting member 111 exerts a “middle” force (a light press). Alternatively, the medical practitioner slides his/her fingertip downward on the press-sensing member 211 so as to generate yet another force-intensity-controlling information C, according to which the pressure-exerting member 111 exerts a “superficial” force (a light touch). Accordingly, the pressure-exerting member 111 stimulates multiple levels of palpation force and exerts the stimulated palpation forces on the pulse taking positions of a subject, and thus the pulse information P of the subject can be detected accurately. It goes without saying that the present invention is not limited to this. The force-intensity-controlling information C also can be generated according to the pressing force exerted on the press-sensing member 211 by the medical practitioner. The pressure-exerting member 111 simulates the pressing force received by the press-sensing member 211, and by this means the pulse information P of the subject can also be detected accurately.

In the pulse-palpating apparatus 100 according to the first embodiment of the present invention as shown in FIG. 3, the network-connected controlling device 2 includes an analyzing means. After the pulse information, such as pulse rate, pulse volume, is stored by the network-connected controlling device 2, the analyzing means 23 analyzes the pulse information P and generates an analysis result according to the rules of pulse diagnosis in TCM. The analysis result includes the pulse pattern of the subject, the physical condition of the Zang-Fu (the internal organs), the advice for the subject, etc., for the medical practitioner's reference. Furthermore, the subject becomes aware of his/her physical condition according to the analysis result generated by self-pulse palpation.

The above description should be considered as only the discussion of the preferred embodiments of the present invention. A person having ordinary skill in the art may make various modifications to the present invention. However, those modifications still fall within the spirit of the present invention and the scope defined by the appended claims.

What is claimed is:

1. A pulse-palpating apparatus, comprising:
a pulse-palpating device, including a pressure-exerting detecting means and a pulse-palpating-end communicating means which is in signal connection with the pressure-exerting detecting means, in which the pressure-exerting detecting means includes a pressure-exerting member and a detecting member in such a manner that after the pressure exerting member exerts a force on pulse taking positions on a human body, the detecting member detects pulse information on the pulse taking positions; and
a network-connected controlling device, including a network-connected controlling means and a controlling-end communicating means, in which the network-connected controlling means is configured to generate force-intensity-controlling information, and the controlling-end communicating means transmits the force-intensity-controlling information to the pulse-palpating-end communicating means via network transmission between the pulse-palpating device and the network-connected controlling device, and receives the pulse information of the detecting member via network transmission,
wherein the force exerted by the pressure-exerting member corresponds to the force-intensity-controlling information.

2. The pulse-palpating apparatus as claimed in claim 1, wherein the quantity of the pressure exerting-detecting means is three, and each pressure-exerting detecting means respectively corresponds to any one of the three pulse taking positions of Cun, Guan, and Chi on the human body.

3. The pulse-palpating apparatus as claimed in claim 2, wherein the pressure-exerting member includes a bulging body that contacts the detecting member, which contacts the three pulse taking positions of Cun, Guan, and Chi on the human body.

4. The pulse-palpating apparatus as claimed in claim 1, wherein the pressure-exerting detecting means is plural, and the pulse-palpating device includes a switching means that is electrically connected between the plurality of pressure-exerting detecting means and the pulse-palpating-end communicating means.

5. The pulse-palpating apparatus as claimed in claim 1, wherein the pulse-palpating device includes a signal converter that is in signal connection between the detecting member and the pulse-palpating-end communicating means so as to convert analog signals of the pulse information to digital signals so as to transmit by the pulse-palpating-end communicating means.

6. The pulse-palpating apparatus as claimed in claim 1, wherein the network-connected controlling means includes a press-sensing member that generates the force-intensity-controlling information according to a pressing manipulation.

7. The pulse-palpating apparatus as claimed in claim 1, wherein the controlling-end communicating means and the pulse-palpating-end communicating means are wirelessly connected.

8. The pulse-palpating apparatus as claimed in claim 1, wherein the network-connected controlling device includes an analyzing means that generates an analysis result according to the pulse information.
